(19) United States
(12) Patent Application Publication
(10) Pub. No.: US 2010/0114181 A1
(43) Pub. Date: May 6, 2010

(54) FIXING ELEMENT FOR A BONE FRAGMENT

(76) Inventor: Günter Lob, Munich (DE)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 12/312,411

(22) PCT Filed: Nov. 13, 2007
(86) PCT No.: PCT/EP2007/062271

§ 371 Date: Jan. 7, 2010

(30) Foreign Application Priority Data
Nov. 15, 2006 (DE) 102006054534.6

(51) Int. Cl. A61B 17/84

(52) U.S. Cl. 606/326

(57) ABSTRACT

A fixing element, in particular a peg, for fixing bone fragments or the like has at least two outer sub-elements extending parallel to each other and an expansion element. The sub-elements and expansion element are of such a configuration and so arranged relative to each other that the fixing element is to be transferred by means of the expansion element from a compact condition into an expanded condition. The two sub-elements define a cavity for the expansion element and have sliding surfaces for the expansion element. The sliding surfaces extend parallel to the longitudinal axis of the fixing element and, in the compact condition of the fixing element, are at a mutual spacing smaller than the mutual spacing of sliding surfaces of the expansion element, that also extend parallel to the longitudinal axis of the fixing element. The sliding surfaces of the sub-elements and the sliding surfaces of the expansion element are of such a configuration and so arranged relative to each other that introduction of the expansion element into the cavity between the sub-elements drives the sub-elements apart in the lateral direction and transfers the fixing element into its laterally expanded condition. The sub-elements provide to the fixing element in its compact condition a cylindrical basic shape of a diameter of between 3 and 7 mm and a length of over 60 mm. The fixing element in its compact condition has longitudinal portions of round cross-section and longitudinal portions of stellate cross-section which alternate in the longitudinal direction of the fixing element. The longitudinal portions of stellate cross-section are provided with prismatic ribs on the outside thereof. The diameter of the longitudinal portions of round cross-section is smaller than the diameter of the longitudinal portions of stellate cross-section.

### BACKGROUND OF THE INVENTION

1. Field of the Invention

The invention concerns a fixing element, in particular a peg, for fixing for example bone fragments, that is to say a fixing element for surgical use.

2. Description of the Background Art

The fixing element is divided in the longitudinal direction into at least two sub-elements and has an expansion element. Those elements are of such a configuration and are so arranged relative to each other that the fixing element is to be converted from a compact condition into an expanded condition by means of the expansion element. For that purpose, the two sub-elements enclose a cavity for the expansion element, the cavity having sliding surfaces for the expansion element, that extend parallel to the longitudinal axis of the fixing element. In the compact condition of the fixing element, the sliding surfaces are at a mutual spacing smaller than the mutual spacing of sliding surfaces of the expansion element, that also extend parallel to the longitudinal axis of the fixing element. That structure provides that insertion of the expansion element into the cavity between the sub-elements causes the sub-elements to be driven apart in the lateral direction and the fixing element to assume its laterally expanded condition.

Such a fixing element is known for example from WO 96/16607.

Fixing elements of that kind are used for example in the manner of a peg for fixing bone elements. For that purpose, the fixing element is inserted into a bore and then expanded by means of the expansion element. By virtue of the expansion effect the fixing element is fixed to the two bone fragments and in that way also fixes the two bone elements relative to each other.

The known fixing element however is not suitable for treating a femur neck fracture. That involves joining the head of the femur (caput femoris) to the shank of the femur (corpus ossis femoris). That is usually effected by way of a connecting element, for example one or more screws, which extends from the shank through the neck (collum femoris) into the head of the femur.

### SUMMARY OF THE INVENTION

The object of the invention is to provide a fixing element of the kind known from WO 96/16607, which has an extended area of use and which is improved in terms of its handling.

According to the invention that object is attained by a fixing element of the kind set forth in the opening part of this specification, which in its compact condition is of a cylindrical basic shape of a diameter of between 3 and 7 mm and a length of over 60 mm. In the longitudinal direction of the fixing element, longitudinal portions of round cross-section and longitudinal portions of stellate cross-section alternate. The stellate cross-section of the corresponding longitudinal portions is formed by prismatic longitudinal ribs on the outside of those longitudinal portions. The diameter of the longitudinal portions of round cross-section is smaller than the diameter of the longitudinal portions of stellate cross-section.

Inserting the expansion element which is of an oversize in relation to the cavity enclosed by the sub-elements means that the two sub-elements are driven apart in parallel relationship by a distance exactly predetermined by the oversize of the expansion element.

Such a fixing element can advantageously be introduced in its compact condition into a round bore which extends from the outside of the bone through the fracture surface into a further bone portion. When the fixing element according to the invention is introduced into such a bore from the outside, it can then be expanded by means of the expansion element and in that way fixes the two bone portions together across the fracture surface. In that respect, it is provided that the fixing element is of an overlength and thus projects proximally out of the bore. The projecting portion of the fixing element can then be cut off.

Advantageously, the fixing element comprises a thermoplastic material so that the fixing element can be cut off by means of a hot wire. In the best-case scenario that additionally entails welding between the outer sub-elements of the fixing element and the expansion element.

Equally, it is advantageous if the material making up the elements of the fixing element is bioresorbable so that the fixing element is gradually absorbed by the body after healing of the fracture. A suitable biocompatible material is for example a polylactide.

The fixing element can in addition advantageously contain osteosynthesis-stimulating substances such as for example bone morphogenic proteins which further promote bone growth. Ideally, the material of the fixing element is so optimized that the delivery of such bone growth-promoting substances is effected in a suitable dose for the desired healing process.

In regard to the external geometry of the fixing element, it is desirable if the longitudinal portions of round cross-section are of a length of between 0.5 and 1.5 mm while the longitudinal portions of stellate cross-section are preferably of a length of between 1 and 3 mm. That geometry permits effective clawing engagement of the expanded fixing element in the respective bone.

In regard to the internal geometry of the fixing element, it is desirable if both the cavity and also the expansion element are of a substantially rectangular cross-section. In that case, the substantially rectangular cross-section of the expansion element is preferably rounded off at the corners. In the longitudinal direction, the expansion element distally has a longitudinal portion which narrows towards the distal end and which allows the expansion element to be introduced with that longitudinal portion leading into the cavity between the two outer sub-elements of the fixing element when the latter is in its compact condition.

It is further advantageous if the separating surface between the two outer sub-elements projects in such a way that even in the expanded condition of the fixing element the two sub-elements are at least not displaceable relative to each other in the longitudinal direction and if possible also not in the lateral direction. For that purpose one of the two sub-elements preferably has projections which protrude beyond a main separating surface and which engage into corresponding recesses in the respective other sub-element. Such projections are preferably associated with each third longitudinal portion of stellate cross-section.

### BRIEF DESCRIPTION OF THE DRAWINGS

The invention will now be described in greater detail by means of an embodiment by way of example with reference to the Figures in which:

FIG. 1 is an exploded perspective view of the fixing element with its two outer sub-elements and the expansion element,

FIG. 2 is a perspective view of the fixing element in its compact condition, and

FIG. 3 is a perspective view of the fixing element in its expanded condition.

### DETAILED DESCRIPTION OF THE INVENTION

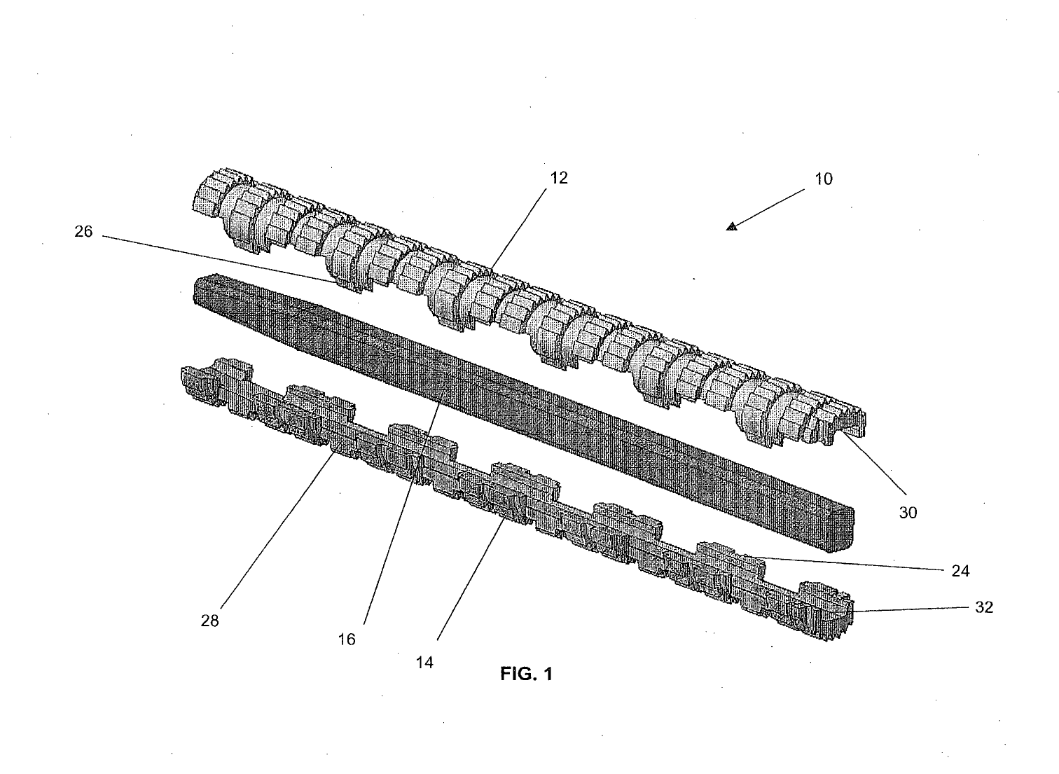

Referring to FIG. 1, therein illustrated is a preferred embodiment of a fixing element 10 made in accordance with the invention. This fixing element 10 has two outer sub-elements 12 and 14 which enclose an expansion element 16.

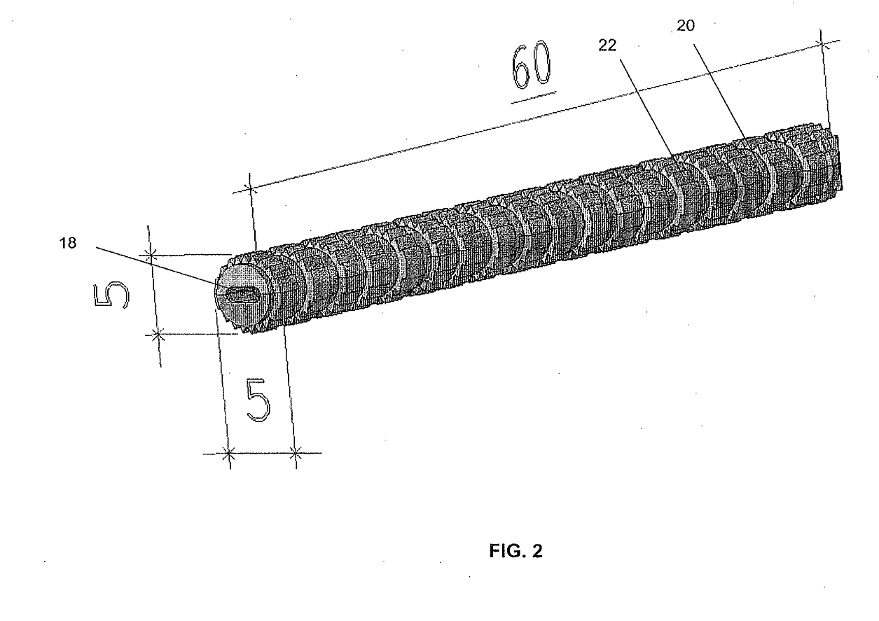

In FIG. 2, the fixing element 10 is shown in its compact condition in which the two outer sub-elements 12 and 14 bear directly against each other. It will be seen that the two sub-elements 12, 14 define a cavity 18 into which the expansion element 16 can be introduced.

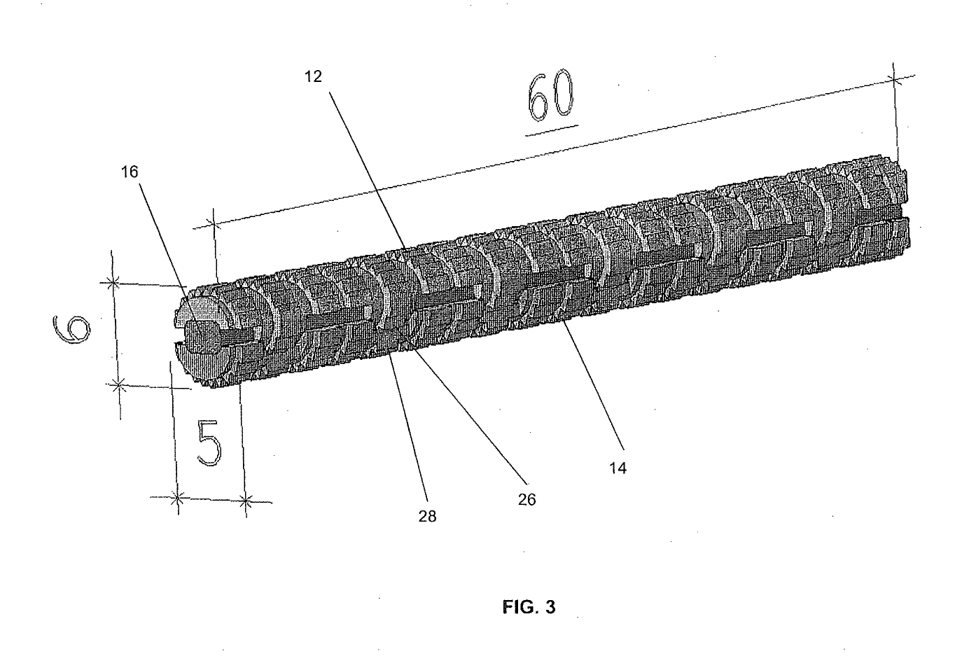

In FIG. 3, the fixing element 10 is shown in its expanded condition with the expansion element 16 has been pushed into the cavity 18.

The two outer sub-elements 12 and 14 are of such a configuration that in the compact condition of the fixing element 10, as shown in FIG. 2, they impart thereto a cylindrical basic shape in which longitudinal portions 20 of round cross-section and longitudinal portions 22 of stellate cross-section alternate. In the illustrated embodiment, the overall fixing element 10 is of a length of 60 mm with a maximum diameter in the region of the longitudinal portions 22 of stellate cross-section of 5 mm. It will be seen that the diameter of the longitudinal portions 20 of round cross-section is somewhat smaller than the diameter of the longitudinal portions of stellate cross-section. As can also be seen from FIG. 2, the stellate cross-section of the longitudinal portions 22 is derived from prismatic longitudinal ribs on the outside of those longitudinal portions.

The two outer sub-elements 12 and 14 are separated from each other along a separating surface 24 which over large parts thereof extends precisely in the center of the fixing element 10 and is flat. At the level of each third portion 22 of stellate cross-section the separating surface projects in such a way that the one outer sub-element 12 has projections 26 which protrude in the direction of the other sub-element 14 and which engage into corresponding recesses 28 on the other outer sub-element 14. Those projections 26 and recesses 28 are so arranged that the two sub-elements 12 and 14 cannot be displaced relative to each other in the longitudinal direction or in the lateral direction, either in the compact condition of the expansion element 10 (see FIG. 2) or in the expanded condition (see FIG. 3) of the expansion element 10.

Depressions 30 and 32 respectively extending along the longitudinal direction of the two outer sub-elements 12 and 14 respectively define the cavity 18 between the two outer sub-elements 12 and 14 into which the expansion element 16 can be inserted. As can be seen from the Figures, essentially over its entire length the cavity 18 is of a uniform rectangular cross-section which is somewhat rounded off at the corners. The cavity 18 narrows somewhat in the region of the distal end of the fixing element 10.

The expansion element 16 is of a cross-section substantially corresponding to the cross-section of the cavity 18. However, the extent of the expansion element 16 in the direction extending transversely relative to the separating surface 24 is somewhat greater than the corresponding extent of the cavity 18 so that the expansion element 16 also actually expands the fixing element 10 when it is pushed into the cavity 18. So that the expansion element 16 can be readily pushed into the cavity 18, even when the fixing element 10 is initially in its compact condition, the corresponding extent of the expansion element 16 continually decreases in the region of the distal end to a dimension which allows the expansion element 16 to be introduced with its distal end leading into the cavity 18 of the fixing element 10 in the compact condition thereof.

The two outer sub-elements 12 and 14 and the expansion element 16 each comprise a bioresorbable thermoplastic material, preferably a polylactide, having the property that after implantation it is gradually absorbed by the human body so that, after a fracture which was fixed by means of the fixing element 10 has healed, there is no longer any implant that has to be removed from the human body. In addition, the material of the two outer sub-elements 12 and 14 and the expansion element 16 has the property that it can be cut with a hot wire so that the fixing element 10 can be of an overlength in relation to a bore in a bone and in that way can be cut off after implantation and insertion of the expansion element 16. A portion of the fixing element 10, that projects externally beyond the bone, can thus be easily cut off. The cutting-off operation is preferably effected with a device having an electrically heated cutting wire with which the thermoplastic material of the fixing element 10 can be easily cut. That advantageously involves a weld between the two outer sub-elements 12 and 14, and the expansion element 16, in the region of the cutting location.

The material of the two outer sub-elements 12 and 14 and optionally also of the expansion element is furthermore preferably provided with a bone growth-stimulating substance which the fixing element 10 after implantation thereof delivers in a dose which is suitable for stimulating bone growth in a manner such as to promote the healing process.

Variants (not shown) of the fixing element according to the invention differ from the fixing element 10 shown in FIGS. 1 through 3 in particular in regard to their length which can be greater than that of the illustrated fixing element 10. Minor deviations in diameter are also possible. Thus it can be up to 1 mm smaller or up to 2 mm larger, than the diameter of the illustrated fixing element. Overall however the fixing element shown in FIGS. 1 through 3 is of a geometry which is advantageously substantially optimized for the intended use, for example for treating femur neck fractures.

1-11. (canceled)

12. A fixing element for fixing bone fragments or the like, comprising at least two outer sub-elements extending parallel to each other and an expansion element, which are of such a configuration and so arranged relative to each other that the fixing element is to be transferred by means of the expansion element from a compact condition into an expanded condition, wherein the two sub-elements define a cavity for the expansion element, the two sub-elements have sliding surfaces for the expansion element within the cavity, the sliding surfaces extend parallel to the longitudinal axis of the fixing element and, in the compact condition of the fixing element, are at a mutual spacing smaller than the mutual spacing of sliding surfaces of the expansion element that also extend parallel to the longitudinal axis of the fixing element, wherein the sliding surfaces of the sub-elements, and the sliding surfaces of the expansion element are of such a configuration and so arranged relative to each other that introduction of the expansion element into the cavity drives the sub-elements apart in the lateral direction and transfers the fixing element into its expanded condition, and wherein the sub-elements provide to the fixing element in its compact condition a cylindrical basic shape of a diameter of between 3 and 7 mm and a length of over 60 mm, the fixing element in its compact condition has longitudinal portions of round cross-section and longitudinal portions of stellate cross-section which alternate in the longitudinal direction of the fixing element, the longitudinal portions of stellate cross-section are provided with prismatic longitudinal ribs on the outside thereof, wherein the diameter of the longitudinal portions of round cross-section is smaller than the diameter of the longitudinal portions of stellate cross-section.

13. A fixing element as set forth in claim 12, wherein the longitudinal portions of round cross-section are of a length of between 0.5 and 1.5 mm.

14. A fixing element as set forth in claim 13, wherein the longitudinal portions of stellate cross-section are of a length of between 1 and 3 mm.

15. A fixing element as set forth in claim 12, wherein the longitudinal portions of stellate cross-section are of a length of between 1 and 3 mm.

16. A fixing element as set forth in claim 12, wherein the longitudinal portions of round cross-section are of a cylindrical shape.

17. A fixing element as set forth in claim 12, wherein the cavity and the expansion element are each of a rectangular cross-section.

18. A fixing element as set forth in claim 17, wherein the expansion element is of a cross-sectional dimension which over the major part of the length of the expansion element remains the same and is larger than the cross-sectional dimension of the cavity in the corresponding direction.

19. A fixing element as set forth in claim 18, wherein, at its distal end, the expansion element (16) is pointed in a wedge shape and narrows in the distal direction to a cross-sectional dimension smaller than the cross-sectional dimension of the cavity in the compact condition of the fixing element.

20. A fixing element as set forth in claim 12, wherein the fixing element is made of bioresorbable plastic material.

21. A fixing element as set forth in claim 20, wherein the bioresorbable plastic material is or contains a polylactide.

22. A fixing element as set forth in claim 20, wherein the bioresorbable plastic material is a thermoplastic material which is adapted to be cut by means of a hot wire.

23. A fixing element as set forth in claim 12, wherein the fixing element is provided with at least one osteosynthesis-stimulating substance.
